(19) United States
(12) Patent Application Publication
(10) Pub. No.: US 2009/0142425 A1
(43) Pub. Date: Jun. 4, 2009

(54) Physiologically active composition

(76) Inventors: Ralf Jager, Milwaukee, WI (US); Hans-Henning Wenk, Freising (DE); Heike Tom Dieck, Freising (DE); Hans Ullrich Hoppe, Lagenbach (DE); Roland Rabeler, Munchen (DE)

Correspondence Address: FULBRIGHT & JAWORSKI, LLP, 666 FIFTH AVE, NEW YORK, NY, 10103-3198, US

(21) Appl. No.: 11/658,342

(22) PCT Filed: Jul. 21, 2005
(86) PCT No.: PCT/EP2005/007964

§ 371 Date: Sep. 3, 2008

(30) Foreign Application Priority Data
Jul. 24, 2004 (DE) 10 2004 036 047.2

(51) Int. Cl. A61K 31/12; A61K 31/09; A61K 31/352; A61K 31/351; A61K 31/7028; A61K 36/00; A61K 36/886; A61K 31/20; A61K 31/66; A61P 3/10; A61P 3/00

(52) U.S. Cl. 424/744; 514/679; 514/718; 514/456; 514/460; 514/25; 424/725; 514/558; 514/75

(57) ABSTRACT

A physiologically active composition is claimed which comprises effective amounts of at least one compound from the group 4-O-methyldavidigenin, 4′-O-methyldavidigenin, davidigenin, elemicin, isoelemicin, herniarin, demethoxycapillarisin, in particular 6-demethoxycapillarisin and/or 6-demethoxy-3′-methoxy-capillarisin, hispiludin and 9-hydroxy-10E, 12Z, 15Z-octadecatrienoic acid, and also glycosides thereof, in particular glucosides and/or rhamnoglucosides of 4-O-methyldavidigenin and/or 4′-O-methyldavidigenin, salts and derivatives.

The present invention relates to a physiologically active composition containing effective amounts of defined compounds, and also to use thereof.

Compositions which contain natural extracts or principal active compounds present therein are being increasingly frequently used by consumers for self-medication of metabolic disorders. In this case there is confidence in particular in plant-based extracts which are known for example from naturopathy or which are due to demonstrated effects in foreign cultural circles.

Typical diseases of the population are cardiovascular diseases and also metabolic diseases such as, in particular, diabetes and its precursors and also pathologically changed blood values which are due to inadequate nutrition or insufficient movement, with mention being made in particular of increased cholesterol or blood fat values.

In particular in connection with metabolic diseases, in the interim numerous formulations are available without prescription on the market, with increasing interest being in the prevention and treatment of diabetes, and in particular diabetes type 2.

From the prior art a plurality of documents are known which encompass the treatment of diabetes using natural extracts or plant components.

For instance, international patent application WO 00/15174 A2 describes the use of bioflavonoids of defined composition, also in the form of a plant extract, for decreasing the blood glucose level of mammals. Typical bioflavonoids which are mentioned are hesperidin, hesperetin, naringin, naringenin, diosmin, rutin and quercetin.

European patent application EP 0 902 870 A1 likewise discloses, inter alia, naringenin and naringin as suitable flavanones for lowering the blood glucose or the fat level, or else for prevention of diabetes and hyperlipidemia. In this context it is also disclosed that flavanones can originate from plant extracts such as, for example, citrus fruits.

According to the publication in Herba Hungarica 1987, Tom. 26, No. 1, flavonoids such as are present in differing organs of selected Bauhinia species, can exhibit an effect on the blood glucose level. In this context, mention is made in particular of quercitrosides and kaempferol-3-galactosides and also -rhamnoglucosides; however, 5,7-dimethoxyflavanones and also 4-O-L-rhamnopyranosil-β-D-glycocyranosides are also mentioned.

Finally, international patent application WO 03/020026 A1 claims different methods for affecting the blood glucose level, the activity of glucagon-like peptide 1 (GLP1), the insulin-dependent glucose intake or else also methods for treatment of type 2 diabetes, generally effective doses of a moderately polar extract of Artemisia species, and in particular of Artemisia dracunculus, needing to be taken.

Diabetes is generally taken to mean a complex metabolic profile or disease which in most cases is characterized by an increased blood sugar level, also being accompanied by serious effects on the metabolism of carbohydrates, fats and proteins. This disease profile results from the inability to control the blood sugar level which is due, for example, to an unsatisfactory insulin level (absolute insulin deficiency) or an inadequate insulin activity (relative insulin deficiency). Increased glucose levels in turn lead to secondary health problems which make additional treatment steps necessary. The chief risk factors which are considered to be associated with diabetes are arteriosclerosis, diabetic retinopathy, cataract, nephropathies, increased infection hazards, high blood pressure, nervous disorders, but also dementia.

In total, a distinction is made between two main diabetes types with numerous variations. Type 1 diabetes generally effects infants or youths and results from the inability of the body to produce insulin (absolute insulin deficiency). Type 2 diabetes, which greatly exceeds type 1 diabetes in frequency, is based either on a reduced insulin secretion, or more frequently on insulin resistance (relative insulin deficiency). The conventionally employed treatment methods are directed toward lowering the blood sugar level by promoting the non-selective glucose uptake into cells. This little-controlled glucose uptake, however, also affects the adipocytes, as a result of which in turn a weight problem can result. Therefore, in these cases, strict control of the treatment method and the changes achieved thereby is of importance. The most widespread conventional treatment method, that is administration of insulin, remains very expensive and for the patients also still associated with inconveniences and side effects such as the risk of hypoglycemia as a result of overdose, allergic reactions and also development of a local lipodystrophy at the injection sites.

Opposing conventional treatment methods are over-the-counter (OTC) medications, such as the abovementioned natural products, in particular those which are plant-based. One of the numerous plant families which are used in traditional naturopathy is the Artemisia family with over 400 species. Yazdanparast et al. in Biomedical Letters 59 (1999), pages 137 to 141, reported of Artemisia dracunculus that alcohol-based extracts thereof are able to exert antihyperlipemic effects on rats. As described extensively in the abovementioned PCT application WO 03/020026, however, beneficial effects in connection with a disturbed fat or sugar balance have also been reported for other Artemisia species such as, for example, A. herba-alba or A. judaica.

The general problem with natural extracts is the standardization, which can only be carried out with difficulty, especially since the cultivation and harvesting conditions, and also storage conditions and the type of workup have significant effects on the components and their respective fraction in the starting material and also in the extract obtained therefrom. It is known that the origin and in particular the associated climatic factors and soil quality significantly affect quality and quantity of the plant components. However, effects such as storage temperature, action of air and moisture on the already harvested material also play a role, for example in the form of oxidative effects. In addition, obviously the type of workup and in particular the type and quality of solvents used are of importance, so that even in the case of identical plant species serious differences in the composition of plant extracts may be observed.

This arises in particular in the case of the above-described Artemisia varieties which are representatives of tarragon. What is termed “German” or “French” tarragon is the most aromatic cultivated form and contains up to 3% essential oil, the aroma of which is governed by methylchavicol (estragol) and -eugenol. In addition, p-methoxycinnamic acid, phellandrene, α- and β-pinene, camphene, ocimene and limonene are also present. Another variety, that is “Russian” tarragon, contains significantly fewer essential oils, estragol being absent completely, as a result of which this variety does not develop the otherwise so pleasant sweet aroma of the French tarragon. The flavonoids which are present instead, quercetin and patuletin, are distinguished by a bitter and astringent taste. Russian tarragon is therefore not customarily described as a typical culinary herb, but is rather assigned to the original wild types of tarragon. Russian tarragon, however, may be more readily cultivated in a relatively cold climate. A survey of potential components of Artemisia varieties and, in particular, Artemisia dracunculus may be found in the survey of the Agricultural Research Services (“Phytochemical and Ethnobotanical Databases”) (http://sun/ars-grin.gov:8080/npgspub/xsql/duke/plantdisp.xsql?taxon=123)

In the already repeatedly cited international patent application WO 03/020026 A1, typical representatives of components are listed which can also be present in Artemisia extracts. For instance, by way of example capillarisin, tetrahydroxymethoxyflavanones, umbelliferons, sakuranin, trihydroxymethoxyflavanones and trihydroxyflavanones are listed. This document also discloses various chromatograms of fresh and frozen Artemisia extracts, in which, however, the peaks given in each case do not permit any conclusions to be drawn about defined compounds possibly present, although in the examples estragol and methyleugenol are mentioned as typical components of Artemisia dracunculus extracts. Owing to the statement which is likewise made that in completely grown plants the biomass ratio shifts from the leaves to the stem, accordingly, the extracts studied in accordance with this document principally proceeded on separated stems and leaves. The preferred extract profiles were prepared on the basis of worked-up Artemisia leaves, with only a relative evaluation of the extracts examined in more detail having been performed. For instance, it is mentioned, for example, that extracts of Artemisia dracunculus which were obtained from frozen plant material displayed a higher activity than fresh plant material. Only on the basis of one comparison with the Wiley registry of mass spectra could main peaks be identified which apparently led to the abovementioned compounds as potential components. Further components which inter alia could be responsible for the co-claimed beneficial action on a disturbed blood sugar level, however, were not identified in the weakly polar ethanolic extracts described.

It was therefore an object of the present invention to provide a physiologically active composition by which these difficulties occurring in the prior art can be overcome, and which consists, in particular of defined compounds or classes of compounds and can be employed in effective doses for the above-described syndromes and symptoms also. This composition should contain as far as possible readily accessible plant material and be readily producible in standardized form. In addition, this composition should consist of components which go beyond the compounds known from the prior art and, in interaction with these, but also with one another, if possible, have synergistic effects with respect to the field of application respectively chosen.

The object has been achieved according to the invention by a corresponding composition which comprises effective amounts of at least one compound selected from 4-O-methyldavidigenin, 4′-O-methyldavidigenin, davidigenin, elemicin, isoelemicin, herniarin, demethoxycapillarisin, in particular 6-demethoxy-capillarisin and/or 6-demethoxy-3′-methoxycapillarisin, hispiludin and 9-hydroxy-10E, 12Z, 15Z-octadecatrienoic acid, and also glycosides thereof, in particular glucosides and/or rhamnoglucosides of 4-O-methyl-davidigenin and/or 4′-O-methyldavidigenin. In addition, their respective suitable salts and derivatives can also be comprised and also all compounds detectable in an Artemisia extract using HPLC analysis.

Surprisingly, it has been found that the claimed compounds can actually be detected in plant extracts which are used as a basis for the production of agents for treating metabolic disorders. In contrast to the previously known publications, these compounds could be observed preferably in Artemisia extracts, contrary to expectations, the respective concentrations occurring in an amount which were not derivable from the previously known analyses. As a further advantage it was observed that the claimed composition is in particular readily accessible such that it is available not only via combination of the pure substances, but also in a simple manner by the selection of suitable plant extracts, the production of these plant extracts themselves not needing to proceed via the previously known work-up methods of plant material.

The claimed composition is also distinguished in that in preferred embodiments, it additionally comprises at least one compound selected from chlorogenic acid, naringenin, flavonoids, flavanoids, terpenes, terpenoids and/or derivatives thereof, and which are taken to mean, in particular, glycosides and/or aglycones. These compounds are not novel as individual substances, but in combination with the components comprised according to the invention also exhibit synergistic effects, preferably additive effects.

According to the present invention, a composition is preferred which comprises the compounds in pure form and/or as a mixture, the mixture preferably then being present as natural extract. On account of their physiochemical properties, the compounds comprised by the composition according to the invention are suitable in particular as alcoholic extracts with respect to their preparation form, preferably alcoholic/aqueous extracts being suitable, and in this content, in particular, their ether fractions.

Alcohols as liquids known to be polar, and especially methanol, ethanol and isopropanol, are outstandingly suitable for producing such extracts which, in the present case as a particular characteristic, omit no mutagenic activities or else contain harmful toxins.

The production of such extracts is in no way limited, but it is advisable first to bring the plant starting material into contact with what is termed an inductor component, for which in particular polysaccharides such as, for example, chitosan, but also acetic acid, methyl salicylate, methyl jasmonate, or even microorganisms, are particularly suitable. Subsequently, the starting material thus digested is treated with the extraction liquid, for example the alcoholic/aqueous solution. The alcohol content should be between 40 and 75%, preferably alcohol contents, of which in particular ethanol contents, between 50 and 60%, having proven very suitable.

In addition, and depending on the starting materials selected, this can also be pretreated by mechanical measures or additionally prepared during the extraction process, by in particular the cell wall and membrane structures being destroyed and thereby the components are released and accessible to the solvent. In particular customary grinding processes should be selected for this. However, it is also possible to carry out the drying of the fresh or frozen and thawed-out starting material at elevated temperatures, in order in this manner to reduce the concentration of methyleugenol which is a component, a recognized carcinogen and which occurs in particular in certain Asteraceae such as also tarragon plants.

The present invention encompasses as preferred in particular compositions in which it is an Artemisia extract. In this case extracts which are particularly suitable are extracts of Artemisia, dracunculus, A. herba-alba, A. judaica, A. vulgaris, A. abysinica, A. absynthicum, A. afra, A. cannariensis, A. pallens, A. annua, A. abrotanum, A. ludoviciana, A. capillaris and A. scoparia.

In the analysis of the components present in these extracts, as particularly suitable subspecies of A. dracunculus those which have particularly proved advantageous are, in particular, those of Russian or French tarragon, since these contain the compounds present as essential to the invention in sufficient and stable concentrations.

In this context, it may be mentioned that the composition according to the invention is advantageously characterized in that it contains the respective compound or mixture or the extract in effective amounts which are between 1 and 1 000 000 ppm, the maximum value characterizing the pure substance. Preferably, however, effective amounts are between 10 and 20 000 ppm, and particularly preferably between 100 and 5000 ppm. These amounts reflect, in particular, the fact that the different compounds exhibit their, in part differing from one another, but supplementary, actions in different amounts, which, as already discussed, can extend from approximately the homeopathic range up to the pure substance.

In addition to the compositions described, the present invention also covers the use thereof for producing an agent for the prevention and/or treatment of (pre) diabetes and thereby associated forms, accompanying disorders and/or secondary disease, with in total eleven fields of application being considered as preferred. Principally the claimed use of the composition for producing an agent with comprises those agents with which preferably a) the blood sugar level in mammals, b) insulin resistance, c) hepatic glucose release, d) the activity of GLP-1 (“glucagon-like-peptide 1”), e) the binding capacity between GLP-1 and the associated receptor, f) the conversion of glucose to glycogen, g) the expression of the IRS-2 (“insulin receptor substrate 2”) polypeptide, h) the insulin-controlled glucose uptake and/or i) the postprandial glucose level are influenced. However, an agent is also suitable which can be used j) for treating or preventing diabetes type 2 and/or 1, k) for treating or preventing pre-diabetes and/or 1) for targeted influencing of bodyweight and/or m) for increasing the physical performance ability of the body.

Said possibilities for influencing metabolic processes can preferably serve for reduction in the case of influencing a) blood sugar level, b) the insulin resistance, c) the hepatic glucose release and/or i) the post-prandial glucose level and take place for their increase in the cases d) of the activity of GLP-1, e) of the binding behavior between GLP-1 and its receptor, f) of the conversion of glucose to glycogen, g) of the expression of the IRS-2 polypeptide, and also h) of the insulin-controlled glucose uptake.

In another embodiment, the present invention encompasses the non-therapeutic use of the agent as food supplement, drink, (dietetic) food and/or functional food. However, its use in the context of clinical nutrition and/or as sport's food is also possible, with in each case the non-therapeutic field of application being in the foreground. The agents which can be produced by the claimed composition are thus suitable in particular for the OTC products and thereby also for self-medication.

Independently of their respective purpose of use, and the application form associated therewith, the present invention provides the use of the agent in amounts in which the effective daily amount of the composition is between 0.1 and 500 mg/kg of bodyweight, with ranges between 1.5 and 150 mg/kg of bodyweight being considered preferred, and 15 mg/kg of bodyweight being considered as particularly preferred.

In addition to the compounds present according to the invention and the additionally listed compounds, the agent can also further comprise at least one active compound and/or one active extract which are selected from Gymnema sylvestre, fenugreek, bitter melon, α-lipoic acid (salts), corosolic acid, ursolinic acid, D-pinitol, Aloe vera, chromopicolinate, banaba, Yacou, Momordica charantia, olive, Pherocarpus marsupium, Salacia reticulata, garlic, hawthorn, phospholipids, and in particular phosphatidylserine, omega-3 fatty acids and starch. The agent thus composed can be used in the context of the present invention, in particular for the prevention and treatment of pre(diabetes).

The present invention, however, also co-comprises the use of an agent which, in addition to the physiologically active composition, comprises at least one active compound selected from pyruvic acid, L-carnitine, hydroxycitric acid, ephedrine, caffeine, conjugated linolic acid, acetylsalicylic acid, α-lipoic acid and/or salts and derivatives thereof. This agent has been shown to be particularly suitable with respect to the control of bodyweight, and in particular for reducing bodyweight.

In the context of the present invention, however, it is also possible to use an agent which, in addition to the physiologically active composition, comprises at least one active compound selected from guanidine compounds, in particular creatine, creatine monohydrate, guanidinoacetic acid, creatinol, creatine-citric acid compound, creatine pyruvate, phosphocreatine, and also caffeine, α-lipoic acid, glucosamine, chondroitin, hydrolyzed collagen, methylsulfonylmethane, whey protein, L-glutamine, phospholipids, in particular phosphatidylserine, phosphatidylcholine, and also choline, β-hydroxy-β-methyl butyrate, pyruvic acid, L-carnitine, D-ribose, amino acids, S-adenosylmethionine, taurine, conjugated linolic acid, glycerol, cinnamon and/or salts and derivatives. This variant of the agent is above all for raising the cell energy in non-adipose cells.

In addition, according to the present invention, a medicament is claimed which uses the composition according to the invention alone or in combination with at least one of the abovementioned active compounds and/or extracts.

Overall, using the claimed composition and the associated application cases, not only could the objective be completely met, but, in particular, defined compounds having a beneficial effect on metabolic processes could be used in combination, these compounds exhibiting their desired effects not only as pure substance, but also in the form of extracts, and in particular plant extracts. The efficacy of these compounds can in part be raised superadditively by combination with one another, but also with other classes of compounds, which, in particular, in connection with self-medication in the non-medical field, brings significant advantages for the end consumer.

The examples hereinafter illustrate the said advantages of the present invention.

### EXAMPLES

### Example 1

### Production of a Tarragon Extract

500 g of dried overground plant parts of Russian tarragon (Artemisia dracunculus) were comminuted (particle size <10 mm) and extracted with 8 l of ethanol 80% by volume for 16 h at 45° C. Subsequently the extract was filtered, the filtrate was concentrated on a rotary evaporator to 200 ml, admixed with 50 g of microcrystalline cellulose and freeze-dried. 175 g of a greenish powder were obtained.

HPLC analysis of the extract gave the following contents:

| | | |
|---|---|---|
| Herniarin | 360 | ppm |
| Davidigenin | 990 | ppm |
| 6-Demethoxycapillarisin | 1450 | ppm |
| 6-Demethoxy-3′-methoxycapillarisin | 15 | ppm |
| Elemicin | 195 | ppm |
| Isoelemicin | 420 | ppm |
| Hispiludin | 65 | ppm |
| 9-Hydroxy-10E,12Z,15Z-octadecatrienoic acid | 25 | ppm |
| 4′-O-Methyldavidigenin | 465 | ppm |
| 4-O-Methyldavidigenin | 35 | ppm |
| 4-O-Methyldavidigenin 4′-glucoside | 585 | ppm |
| 4-O-Methyldavidigenin 4′-rhamnoglucoside | 675 | ppm |

### Example 2

### Acute Effects of the Alcoholic Tarragon Extract on the Post-Prandial Blood Glucose Level in Diabetes Type 2—Animal Models

Hereinafter, the results of animal studies are listed in which the acute effect of a tarragon extract in the post-prandial blood glucose level were studied in animals having obesity and simultaneously increased fasting blood sugar (diabetes type 2). Use was made of a glucose solution which contained a powder obtained according to Example 1.

The investigation time was a max. of 5 hours in each case, in which an oral glucose tolerance test was carried out with every animal either with various doses of the extract or with placebo.

In total 30 rats having an elevated blood sugar level were studied, or ob/ob mice (adipose mice which are homozygotic for the ob gene (from obese)) were studied, with 10 animals being studied per treatment group.

After a fasting period of 14 hours, the fasting blood sugar level was determined in the animals. This was a mean 138.8 mg/dl (mice) or 136.0 mg/dl of glucose (rats) and did not differ significantly between the groups. A defined amount of glucose in solution (3.33 ml of solution/kg of bodyweight, rats, or 10 ml/kg of bodyweight, mice) was subsequently administered to the animals by gastric tube, which solution contained either 500 mg/kg of bodyweight or 1000 mg/kg of bodyweight of the extract solution or, as a control consisted only of the glucose solution (60% strength (rats) or 20% strength (mice)+2% Tween 80 as solubilizer). After administration of the glucose solution, blood was taken at 15 minute intervals for a period of 180 min to determine the blood glucose concentration and insulin concentration. The values of post-prandial glucose levels are given in Tables 1 and 2.

All animals tolerated the treatments without any indications of incompatibility.

TABLE 1
Post-prandial blood glucose concentrations (in mg/dl)
in diabetic mice which were treated with 500 mg/kg
of bodyweight or 1000 mg/kg of bodyweight extract in
glucose solution or control glucose solution.

| Time | | | | | | | |
|---|---|---|---|---|---|---|---|
| [min] | 0 | 15 | 30 | 60 | 90 | 120 | 180 |
| | Control | | | | | | |
| Mean | 142.2 | 433.2 | 686.0 | 357.1 | 340.4 | 335.0 | 313.5 |
| SD | 54.2 | 88.2 | 118.9 | 110.5 | 124.2 | 131.9 | 97.5 |
| (standard | | | | | | | |
| deviation) | | | | | | | |
| | 500 mg/kg of bodyweight | | | | | | |
| Mean | 143.7 | 353.9 | 534.2 | 319.7 | 274.0 | 260.5 | 259.9 |
| SD | 60.4 | 85.7 | 100.2 | 71.8 | 77.9 | 61.5 | 50.7 |
| | 1000 mg/kg of bodyweight | | | | | | |
| Mean | 130.5 | 313.4 | 476.9 | 300.9 | 265.6 | 251.3 | 236.1 |
| SD | 44.6 | 88.1 | 100.9 | 76.8 | 63.8 | 75.4 | 76.6 |

TABLE 2
Post-prandial blood glucose concentrations (in mg/dl)
in diabetic rats which were treated with 500 mg/kg
of bodyweight or 1000 mg/kg of bodyweight extract in
glucose solution or control glucose solution.

| Time | | | | | | | |
|---|---|---|---|---|---|---|---|
| [min] | 0 | 15 | 30 | 60 | 90 | 120 | 180 |
| | Control | | | | | | |
| Mean | 133.2 | 323.7 | 430.2 | 452.6 | 367.5 | 286.3 | 235 |
| SD | 28.3 | 43.8 | 62.3 | 75.9 | 79.3 | 81.7 | 65.5 |
| | 500 mg/kg of bodyweight | | | | | | |
| Mean | 135.2 | 306.9 | 382.4 | 364.2 | 294.8 | 230.5 | 195.8 |
| SD | 16.1 | 65.8 | 77.2 | 59.3 | 58.2 | 55.2 | 46.8 |
| | 1000 mg/kg of bodyweight | | | | | | |
| Mean | 139.6 | 281.4 | 314.3 | 297.7 | 245.6 | 210.4 | 194.2 |
| SD | 42.2 | 99.8 | 82.4 | 91.8 | 102.2 | 93.2 | 55.4 |

### Example 3

### Acute Effects of Tarragon Extract on the Post-Prandial Blood Glucose Levels in Humans Having Glucose Intolerance or Type 2 Diabetes

A monocentric, double-blind randomized placebo-controlled cross-over study for investigating the acute activity of the tarragon extract on the post-prandial blood sugar level in persons having glucose intolerance or type 2 diabetes. The action of the preparation was determined by means of a meal tolerance test (MTT).

8 male or female patients, exclusively treated by diet, having elevated fasting blood sugar levels were included in the study. The study, in addition to a screening test (V0), in which the possible participants were tested for their suitability to take part in the study, comprised two ambulant study days, visit 1 (V1)+visit 2 (V2), separated by a 2- to 3-week wash-out period and also a final investigation subsequent to V2.

After the suitability of the participants had been established, the participants were assigned to a treatment plan by randomization and received both treatments in the course of the study in a double-blind cross-over method, both with extract and also with placebo (microcrystalline cellulose). On the respective study days, the patients in each case received, before a standard breakfast, a single oral administration of either 1000 mg of extract or placebo, after which subsequently the action of the treatment on the post-prandial blood sugar level was determined by regular determination of the blood sugar values and subsequent analysis of the serum insulin levels over a period of 5 hours. With the completion of the visit 2, during which course a final investigation was carried out, the study was terminated for the participants.

A precondition for participation of an interested person in a clinical study was his or her written consent for participation after which he or she was informed orally and in writing on the nature, importance and consequences of the clinical study. The investigators obliged themselves to carry out the study in agreement with the declaration of Helsinki (in the Revision of Edinburgh, October 2000), the principles of Good Clinical Practice (GCP), of the International Conference of Harmonization (ICH) and the national ordinances and guidelines.

The participants were informed with an information leaflet on all aspects of the study and also on data protection. The informing of the subjects, in addition to the consent declaration of the subjects, was documented by signature of the investigating doctor responsible.

In accordance with the inclusion criteria, 5 male and 3 female subjects of ages between 35 and 70 years, a BMI >29 and <40 kg/m², a fasting blood sugar level ≧125 and ≦220 mg/dl, a HbA1c value >6.1% without antidiabetic treatment with tablets or insulin, and also the provision of written declaration of agreement were included in the study. These subjects, according to estimation of the investigating doctor, did not exhibit any clinically significant deviation of laboratory value in the preliminary test (in particular: serum creatinine and serum hemoglobin values, and also activity of glutamate-oxalacetate transaminase (GOT) and glutamate-pyruvate transaminase (GPT), which would indicate acute or chronic disease/disorder).

TABLE 3
Post-prandial plasma glucose concentrations (in
mg/dl) in diabetic subjects treated with 1000 mg
of extract or placebo for a standard breakfast

| Time | | | | | | | |
|---|---|---|---|---|---|---|---|
| [min] | 0 | 30 | 60 | 90 | 120 | 180 | 300 |
| | Placebo | | | | | | |
| Mean | 142.4 | 202.7 | 234.4 | 243.7 | 223.5 | 185.7 | 152.6 |
| SD | 2.59 | 5.95 | 7.08 | 9.53 | 7.32 | 4.89 | 3.30 |
| | 1000 mg | | | | | | |
| Mean | 141.3 | 185.7 | 218.4 | 207.6 | 184.8 | 159.3 | 133.7 |
| SD | 3.34 | 14.59 | 10.55 | 6.18 | 3.3 | 4.78 | 3.45 |

TABLE 4
Post-prandial plasma insulin concentrations (in μU/ml) in diabetic
subjects treated with 1000 mg or placebo for a standard breakfast

| Time | | | | | | | |
|---|---|---|---|---|---|---|---|
| [min] | 0 | 30 | 60 | 90 | 120 | 180 | 300 |
| | Placebo | | | | | | |
| Mean | 14.1 | 79.9 | 149.8 | 173.8 | 178.7 | 148.3 | 56.9 |
| SD | 4.2 | 18.4 | 26.7 | 20.2 | 23.7 | 14.5 | 6.3 |
| | 1000 mg | | | | | | |
| Mean | 13.2 | 72.1 | 134.4 | 156.7 | 145.5 | 118.6 | 42.9 |
| SD | 2.9 | 20.3 | 18.9 | 26.8 | 19.6 | 11.4 | 7.3 |

1-23. (canceled)

24. A method comprising administering a physiologically active composition comprising a therapeutically effective amount of at least one compound is —O-methyldavidigenin, 4′-O-methyl-davidigenin, davidigenin, elemicin, isoelemicin, herniarin or demethoxycapillarisin or a glycoside thereof to prevent or treat (pre)diabetes and associated accompanying diseases or secondary diseases.

25. The method of claim 24, wherein said at least one compound is 6-demethoxy-capillarisin, 6-demethoxy-3′-methoxycapillarisin, hispiludin or 9-hydroxy-10E, 12Z, 15Z-octadecatrienoic acid.

26. The method of claim 24, wherein the glycoside is at least one of a glucoside or a rhamnoglucoside of 4-O-methyldavidigenin or 4′-O-methyldavidigenin, a salt thereof, a derivative thereof or a compound detectable in an Artemisia extract by HPLC analysis.

27. The method of claim 24, wherein said physiologically active composition further comprises at least one compound selected from chlorogenic acid, naringenin, a flavonoid, a flavanoid, a terpene, a terpenoid or a derivative thereof.

28. The method of claim 27, comprising at least one compound in the form of glycoside or and aglycone.

29. The method of claim 24, comprising the physiologically active composition in pure form as a mixture.

30. The method as claimed in claim 29, comprising the mixture in the form of a natural extract.

31. The method as claimed in claim 30, comprising an alcoholic or alcoholic/aqueous extract.

32. The method as claimed in claim 31, comprising an ether fraction of the alcoholic/aqueous extract.

33. The method as claimed in claim 30, wherein the extract is an Artemisia extract.

34. The method as claimed in claim 33, wherein the Artemisia extract comprises an extract of at least one of A. dracunculus, A. herba-alba, A. judaica, A. vulgaris, A. abysinica, A. absynthicum, A. afra, A. cannariensis, A. pallens, A. annua, A. abrotanum, A. ludoviciana, A. capillaris or A. scoparia.

35. The method as claimed in one of claim 33, wherein the extract originates from a subspecies of A. dracunculus.

36. The method as claimed in claim 35, wherein the subspecies is at least one of Russian or French tarragon.

37. The method as claimed in claim 24, wherein the therapeutically effective amount is between 1 and 1,000,000 ppm.

38. The method as claimed in claim 24, wherein at least one of
a) the blood sugar level in a mammal is lowered,
b) insulin resistance is lowered,
c) hepatic glucose release is lowered,
d) the postprandial glucose level is lowered,
e) the activity of GLP-1 (“glucagon-like-peptide 1”) is raised
f) the binding capacity between GLP-1 and the associated receptor is raised,
g) the conversion of glucose to glycogen is raised,
h) the expression of the IRS-2 (“insulin receptor substrate 2”) polypeptide is raised.
or
i) the insulin-controlled glucose uptake is raised.

39. The method as claimed in claim 38 for treating or preventing diabetes type 2, diabetes type 1 or for targeted influencing of bodyweight.

40. A method comprising administering a physiologically active composition comprising a therapeutically effective amount of at least one compound is —O-methyldavidigenin, 4′-O-methyl-davidigenin, davidigenin, elemicin, isoelemicin, herniarin or demethoxycapillarisin or a glycoside thereof to raise the physical performance ability of the body.

41. The method as claimed in claim 24, wherein the composition is a food supplement, a drink, a food, a dietetic food, a functional food or a sport food.

42. The method as claimed in claim 24, wherein the agent is used in an amount which corresponds to an effective daily amount of the composition and is between 0.1 and 500 mg/kg of bodyweight.

43. The method as claimed in claim 24, wherein the agent further comprises
a) at least one active compound or at least one active extract selected from Gymnema sylvestre, fenugreek, bitter melon, α-lipoic acid or a salt thereof, corosolic acid, ursolinic acid, D-pinitol, Aloe vera, chromopicolinate, banaba, Yacou, Momordica charantia, olive, Pherocarpus marsupium, Salacia reticulata, garlic, hawthorn, phospholipids, omega-3 fatty acids or a starch;
b) at least one active compound selected from pyruvic acid, L-carnitine, hydroxycitric acid, ephedrine, caffeine, conjugated linolic acid, acetylsalicylic acid, α-lipoic acid or salts and derivatives therefor,
c) at least one active compound selected from a guanidine compound, caffeine, α-lipoic acid, glucosamine, chondroitin, hydrolyzed collagen, methylsulfonylmethane, whey protein, L-glutamine, phospholipids, choline, β-hydroxy-β-methyl butyrate, pyruvic acid, L-carnitin, D-ribose, amino acids, S-adenosylmethionine, taurine, conjugated linolic acid, glycerol, cinnamon or a salt or derivative thereof.

44. The method as claimed in claim 43, wherein the phospholipid is phosphatidylserine, and the guanidine compound is selected from creatine, creatine monohydrate, guanidinoacetic acid, creatinol, creatine-citric acid compound, creatine pyruvate or phosphocreatine, and the phospholipid is phosphatidylserine or phosphatidylcholine.

45. The method as claimed in claim 43, wherein a) the agent is used for preventing or treating (pre)diabetes, in b) the agent is used for control of bodyweight, and in c), the agent is used to increase cell energy in non-adipose cells.

46. A medicament comprising a therapeutically effective amount of at least one compound is —O-methyldavidigenin, 4′-O-methyl-davidigenin, davidigenin, elemicin, isoelemicin, herniarin or demethoxycapillarisin or a glycoside thereof to prevent or treat (pre)diabetes or an associated accompanying disease or secondary disease.

47. A medicament comprising a therapeutically effective amount of at least one compound is —O-methyldavidigenin, 4′-O-methyl-davidigenin, davidigenin, elemicin, isoelemicin, herniarin or demethoxycapillarisin or a glycoside thereof to prevent or treat (pre)diabetes or an associated accompanying disease or secondary disease and at least one of the active compounds or active extracts of claim 43.
